(19) United States
(12) Patent Application Publication
(10) Pub. No.: US 2013/0195955 A1
(43) Pub. Date: Aug. 1, 2013

(54) Implants Containing BMP-7

(71) Applicants: Johannes Reichert, Queensland (AU); Dietmar W. Hutmacher, Kenmore Hills (AU)

(72) Inventors: Johannes Reichert, Queensland (AU); Dietmar W. Hutmacher, Kenmore Hills (AU)

(21) Appl. No.: 13/724,660

(22) Filed: Dec. 21, 2012

Related U.S. Application Data
(60) Provisional application No. 61/578,480, filed on Dec. 21, 2011.

(51) Int. Cl. A61K 9/00

(52) U.S. Cl. CPC A61K 9/0087
USPC 424/443; 514/8.8

(57) ABSTRACT

Osoteogenic implants, and related compositions, methods and kits. The implants comprise a biodegradable scaffold comprising a polycaprolactone matrix and tricalcium phosphate particles within the matrix. The implants further include a formulation comprising BMP-7.

### FIELD OF THE INVENTION

The present invention relates generally to biodegradable medical implants, and more particularly to biodegradable composite scaffolds for bone generation.

### BACKGROUND

Bone possesses the unique potential for self-repair after injury. Defects exceeding a certain critical size, however, may not heal spontaneously and usually require additional biological stimuli or tissue transplantation. In orthopedic and trauma surgery, extensive bone loss is associated with major technical and biological problems. Bone grafts used to treat bone defects have the desired osteoconductive and osteoinductive properties. These “autografts,” however, have limited availability and are often difficult to access, causing further pain and additional healing time for the patient. Bone graft harvesting may cause donor site morbidity and increase the risk of infection, whereas transplants may also integrate insufficiently and require additional surgeries. This need drives the orthopedic research community to develop bone graft substitutes to augment large-sized defects.

The delivery of recombinant growth factor proteins has emerged as a promising alternative to bone grafting, to promote endogenous repair mechanisms and tissue regeneration. This approach has been translated into routine clinical applications for the treatment of acute fractures and non-unions, as well as spine and dental applications. Currently, bone morphogenetic proteins (BMPs) are applied by use of absorbable collagen carriers, equipped with solubilized protein. However, undesired side effects associated with rapid protein degradation and diffusion from these carriers, concerns over the incremental effectiveness and cost of BMP on fracture healing, and reports on a correlation between extremely high doses of BMP and cancer incidence, necessitate the optimization of drug delivery, regarding both drug quantity and mode of release by carrier systems. Clearly, the physiochemical and biological properties of these growth factors motivate the need to design scaffolds that allow maintenance of protein bioactivity and enhance growth factor retention at the implantation site.

It is reported in U.S. Pat. No. 7,174,282, incorporated herein by reference, that biomaterial scaffolds for tissue engineering perform three primary functions. The first is to provide a temporary function (stiffness, strength, diffusion, and permeability) in tissue defects. The second is to provide a sufficient connected porosity to enhance biofactor delivery, cell migration and regeneration of connected tissue. The third requirement is to guide tissue regeneration into an anatomic shape. It is noted that the first two functions present conflicting design requirements. Specifically, increasing connected porosity to enhance cell migration and tissue regeneration decreases mechanical stiffness and strength, whereas decreasing porosity increases mechanical stiffness and strength but impedes cell migration and tissue regeneration.

U.S. Pat. No. 8,071,007, incorporated herein by reference, provides a method of using Fused Deposition Modeling to construct three-dimensional bioresorbable scaffolds from polycaprolactone (PCL) and from composites of PCL and ceramics, such as tricalcium phosphate (TCP) with specific lay-down patterns that confer the requisite properties for tissue engineering applications. The resulting three-dimensional polymer matrix has degradation and resorption kinetics of 6 to 12 months and the capability to maintain a given space under biomechanical stress/loading for 6 months. Incorporation of a bioresorbable ceramic in the bioresorbable, synthetic and natural polymer produces a hybrid/composite material support triggering the desired degradation and resorption kinetics. Such a composite material is said to improve the biocompatibility and hard tissue integration.

In addition, it is known in the field that bone morphogenetic proteins, specifically BMP-7, are useful for bone repair and regeneration. U.S. Pat. No. 7,410,947, incorporated herein by reference, provides that mixing osteogenic protein such as BMP-7 and a non-synthetic, non-polymer matrix such as collagen with a binding agent yields an improved osteogenic device with enhanced bone and cartilage repair capabilities. Not only can such improved devices accelerate the rate of repair, these devices can also promote formation of high quality, stable repair tissue, particularly cartilage tissue. Patents such as U.S. Pat. No. 8,275,594, incorporated herein by reference, suggest that biodegradable and biocompatible polymers can be combined with ceramic materials to form a scaffold in which bone morphogenetic proteins may be incorporated. However, in view of the background in the area of biodegradable osteogenic implants, there is still a need for further advances in this technology to optimally combine the usage of scaffolds and BMP-7 formulations.

### SUMMARY OF THE INVENTION

In one aspect, the present invention relates to an osteogenic implant. The implant comprises a biodegradable scaffold comprising a matrix comprising polycaprolactone, and a formulation comprising BMP-7 that is carried by the scaffold. In certain embodiments, the scaffold comprises ceramic particles. In certain embodiments, the scaffold takes the form of a three-dimensional structure.

In another aspect, the present invention relates to a kit that comprises a biodegradable scaffold comprising a matrix comprising polycaprolactone, and a formulation comprising BMP-7.

In another aspect, the present invention relates to a method of treating a patient by administering an implant of the present invention that comprises a biodegradable scaffold comprising a matrix comprising polycaprolactone, and a formulation comprising BMP-7.

### BRIEF DESCRIPTION OF THE DRAWINGS

FIGS. 1a and 1b are scanning electron micrographs of a cross-section view and a top view, respectively, of a scaffold used in the present invention based on a 0/90° lay-down pattern of fibers, in accordance with an embodiment of the present invention.

FIGS. 2a and 2b are scanning electron micrographs of a cross-section view and a top view, respectively, of a scaffold used in the present invention based on a 0/60/120° lay-down pattern of fibers, in accordance with an embodiment of the present invention.

FIGS. 3a and 3b are scanning electron micrographs of a cross-section view and a top view, respectively, of a scaffold used in the present invention based on a 0/72/144/36/108° lay-down pattern of fibers, in accordance with an embodiment of the present invention.

FIGS. 4a and 4b are photographs of a cross-sectional view and a perspective view, respectively, of a scaffold in accordance with an embodiment of the present invention.

### DETAILED DESCRIPTION OF PREFERRED EMBODIMENTS

The present invention provides osteogenic implants and related methods and kits for bone defect regeneration. Generally, the implants of the present invention comprise a biodegradable scaffold comprising a matrix comprising polycaprolactone (“PCL”), and a formulation comprising BMP-7 that is carried by the scaffold. As used herein, “biodegradable” is used synonymously with “bioerodible,” “bioabsorbable,” and other similar terms that describe materials that degrade in vivo or in aqueous-containing environments. As used herein, formulations are described to be “carried by” scaffolds in that the formulations are placed partially or wholly inside of such scaffolds, are placed or coated on one or more surfaces of such scaffolds, are integrated within one or more components of the scaffolds, or are otherwise entrapped, suspended, or bound to the scaffolds. As used herein, “scaffolds” mean structures or materials that are intended to be placed in or on an animal body and that are useful for the purpose of carrying therapeutic formulations. As used herein, “formulation” means a composition that includes an active agent and optionally includes other supplemental materials. As used herein, “BMP-7” means Bone Morphogenic Protein-7, also known as OP-1, including all variants, mutations, peptides and genes thereof.

The inventors have surprisingly found that use of the implants of the present invention result in significantly better bone regeneration results than the use of autografts. It is believed that the unique combination of PCL-based scaffolds with BMP-7, as described herein, result in unexpected and beneficial results when compared with conventional bone treatment methods. Examples of such results are described in J. Reichert et al., “A Tissue Engineering Solution for Segmental Defect Regeneration in Load-Bearing Long Bones,” Science Translational Medicine, 4, 141ra93 (2012), which is incorporated herein by reference.

Scaffolds of the present invention comprise matrix materials that comprise PCL. In preferred embodiments, semi-crystalline PCL having an average Mₙ of about 80,000 Daltons (or 80 kD) is used in the matrix materials of the present invention. The matrix material is preferably formed into fibers using known fiber spinning techniques, with such fibers having diameters preferably within the range of 260-370 microns, more preferably about 300 microns. Such fibers are preferably layered into repeating patterns using a fused deposition modeling (FDM) process, as described in U.S. Pat. No. 8,071,007, which is incorporated herein for all purposes. Various “lay-down patterns” of fibers are possible using the FDM technique, giving rise to complex 3D geometrical patterns as possible scaffold structures. The structure of scaffolds designed and fabricated using the FDM method may be similar to the honeycomb of a bee, with its regular array of identical pores. The main difference lies in the shape of the pores: the bee's honeycomb comprises hexagonal pores surrounded by solid faces/walls which nest together to fill a plane, whereas the FDM scaffold structure is built from inter-crossing filaments stacked in horizontal planes and comprises pores surrounded by solid edges/struts.

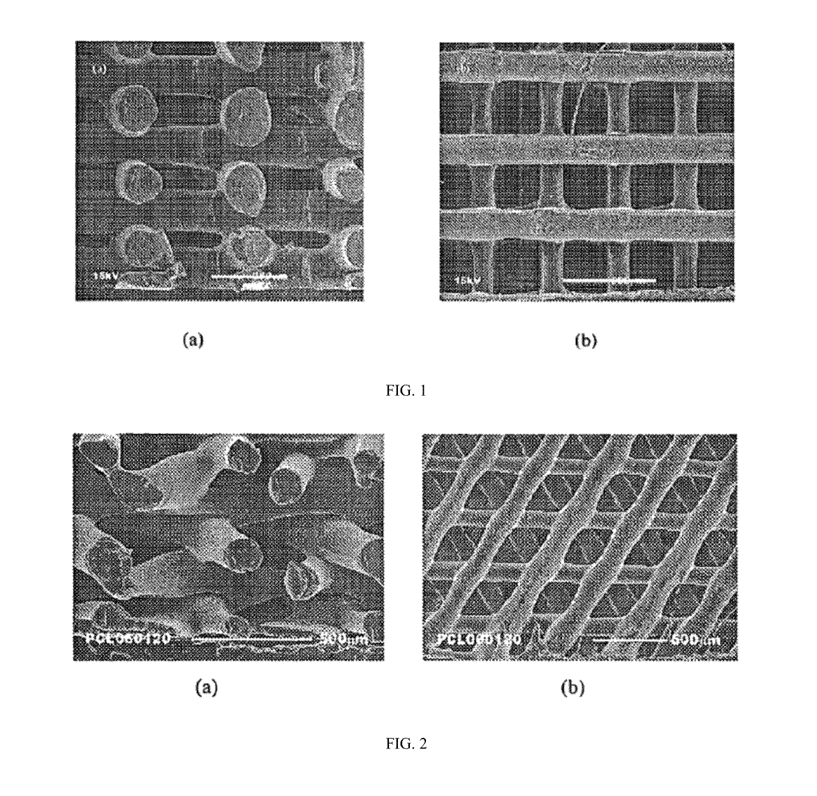

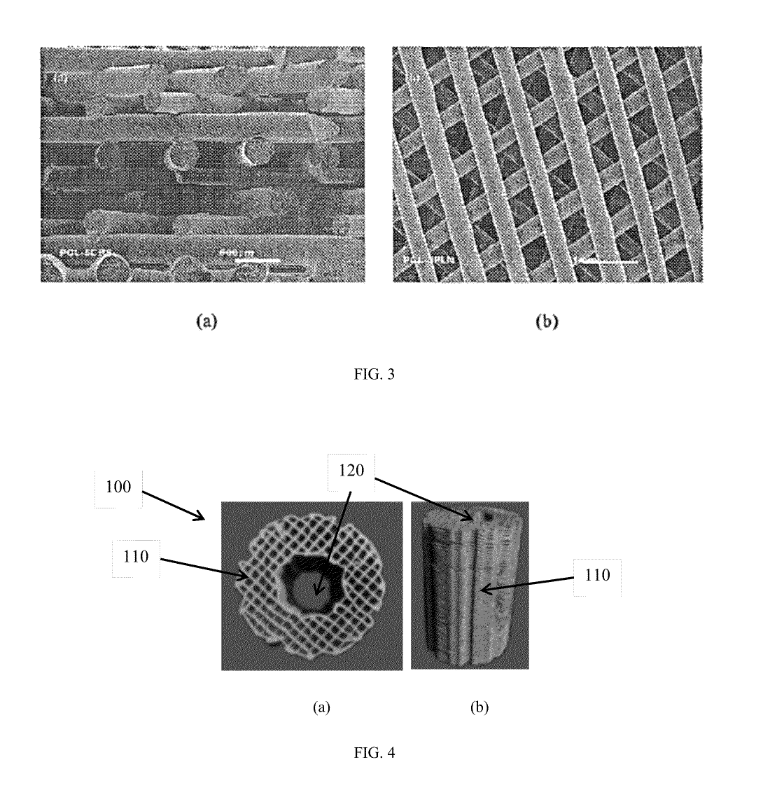

A 0/90° lay-down pattern results in square pores, as shown in FIG. 1. Lay-down patterns of 0/60/120° and 0/72/144/36/108° are used to give a honeycomb-like pattern of triangular and polygonal pores, respectively. These three lay-down patterns are observed in FIGS. 1-3, respectively. The size of the pores (i.e., the space between fibers) can range from 0 to 1200 microns, but is preferably in the range of 200-700 microns, and more preferably 350-550 microns. The amount of porosity in the scaffolds of the present invention is preferably 30%-80%, and more preferably about 70%, and the porosity if preferably interconnected.

The fiber arrangement is structured into a three-dimensional scaffold structure having a size and shape that is suitable for a particular clinical use. For example, the structure may be formed into the shape of nails, pins, screws, plates and anchors for implantation at a bone site. In other examples, the structure may be formed into the shape of long bone segments, bone defects, or interbody spine fusion cages for implantation. In a preferred embodiment, however, the scaffold 100 is a tubular structure 110 having a central lumen 120 therethrough, as shown in FIG. 4. In a preferred embodiment, the scaffold is in the configuration shown in FIG. 4 and has an outer diameter of about 20 mm, a height of about 30 mm, and an inner diameter of about 8 mm. It should be appreciated, however, that the scaffolds of the present invention may or may not have a lumen or other exact configuration, and any lumens within the scaffolds may extend only partially through the scaffold structure. The scaffolds may be implanted into a patient alone or in conjunction with orthopedic implants such as bone plates, such that the scaffolds come into proximity or contact with bone to provide a therapeutic effect. As such, the scaffolds of the present invention are preferably characterized by mechanical properties that facilitate such use. In one example, the scaffold is load-bearing and is characterized by an elastic modulus of 20-25 MPa. In another example, the compression stiffness of scaffolds of the present invention range from 4 to 77 MPa when tested in air and are therefore comparable to human trabecular bone. The inventors have noted that the mechanical properties of scaffolds of the present invention are within the lower range of cancellous bone at scaffold porosities of 60-70%.

Although the scaffolds of the present invention are biodegradable, the degradation time in vivo is relatively long. For example, the in vivo degradation time of the scaffolds of the present invention may range from six months to twelve months to three years.

In certain embodiments of the present invention, the scaffolds comprise ceramic particles embedded therein. As used herein, particles are said to be “embedded” within scaffolds in that they are carried by the scaffold, such as being located within the scaffold pores or are coated on one or more surfaces of the scaffold. The amount of ceramic particles within the scaffolds is preferably 20-80 weight percent, more preferably 20-50 weight percent, and most preferably about 20 weight percent. In a preferred embodiment, the ceramic particles comprise a calcium phosphate. As used herein, a “calcium phosphate” means a synthetic bone substitute material comprising calcium phosphate as the primary component. Suitable calcium phosphate-based materials are well known in the art and include, without limitation, amorphous apatitic calcium phosphate, hydroxyapatite, and fluorapatite, and more preferably tricalcium phosphate. The ceramic may be amorphous, crystalline, or a mixture of both.

Implants of the present invention comprise a formulation comprising BMP-7 that is carried by the scaffold. BMP-7 is a member of the TGF-β superfamily of proteins that is known for its bone healing and growth properties. For example, OP-1 IMPLANT and OP-1 PUTTY are marketed by Olympus Biotech Corporation (Hopkinton, Mass.) and incorporate BMP-7 as an active agent. OP-1 IMPLANT is an osteoinductive and osteoconductive bone graft material. It is a combination of 3.3 mg of recombinant human BMP-7 (rhBMP-7) and 1 g of purified Type I bovine collagen, which is used as a carrier. The product is reconstituted with 2-3 cc of saline to form a paste which is then implanted at the nonunion site. OP-1 PUTTY is an osteoinductive and osteoconductive bone graft material. OP-1 PUTTY consists of the recombinant human BMP-7 (rhBMP-7), Type I Bovine Bone Collagen Matrix (collagen matrix) and the Putty Additive carboxymethylcellulose sodium (CMC). OP-1 PUTTY is intended to be reconstituted with sterile saline (0.9%) solution.

An effective amount of BMP-7 is used in implants of the present invention. As used herein, “effective amount” means an amount sufficient to stimulate osteogenic activity of present or infiltrating progenitor or other cells.

The formulations of the present invention may be of any suitable form, such as pastes, putties, gels, granules, films, or the like. Preferably, the formulations of the present invention are used in a paste or putty form and applied to the scaffold. In a preferred example, the paste or putty is applied into the lumen 120 of scaffold 100, as shown in FIG. 4. It should be appreciated, however, that the formulations may be carried by a scaffold in any manner; as non-limiting examples, the formulations may be applied to one or more exterior or interior surfaces of the scaffold, or injected into the interconnected porosity of the scaffold.

Formulations of the present invention may optionally include one or more additives or supplemental materials. As known in the art, supplemental materials may be used in therapeutic formulations to improve tensile strength and hardness, increase fracture toughness, provide imaging capability, and the like.

In one embodiment, the formulations of the present invention comprise an effervescent agent as an additive. “Effervescent agent” refers to a gaseous substance or a substance, which produces bubbling, foaming or liberation of a gas. An exemplary effervescent agent is sodium bicarbonate, carbon dioxide, air, nitrogen, helium, oxygen, and argon. Formulations of the present invention may include, for example, from about 1 to about 40 weight percent of an effervescent agent. In other embodiments, the formulations of the present invention comprise binders such as bone glues, cements, fillers, plasters, epoxies, or gels such as, but not limited to, calcium sulfate, alginate, and collagen. In other embodiments, the formulations of the present invention comprise one or more additives that alter resorption properties of the implant.

The present invention includes kits that include scaffolds as described herein and formulations as described herein packaged together in a single or bundled package. The present invention also includes methods of using the scaffolds and formulations of the present invention. The methods optionally include the steps of reconstituting the formulations of the present invention with saline or other liquids, applying the formulation to the scaffold to yield the implant, and implanting the implant into a patient using known surgical techniques to provide a needed therapeutic effect.

The invention will not be more particularly described with reference to the following specific examples. It will be understood that these examples are illustrative and not limiting of the embodiments of the invention.

### EXAMPLE 1

### Scaffold fabrication and preparation

Cylindrical scaffolds of mPCL [number-average molecular weight (Mₙ), 80 kD; 1.145 g/cm³; Sigma-Aldrich] incorporating 20% β-TCP microparticles (Sigma-Aldrich; outer diameter, 20 mm; height, 30 mm; inner diameter, 8 mm) were fabricated by fused deposition modeling (Osteopore International) as described in U.S. Pat. No. 8,071,007, which is incorporated herein by reference for all purposes. Scaffolds were pretreated with 1 M NaOH for 6 hours to render the scaffolds more hydrophilic and were sterilized. Fibers of about 300 μm in diameter were deposited after a 0/90° pattern with a separation of about 1200 μm, resulting in a scaffold with 70% porosity and fully interconnected pores. The scaffolds were characterized by an elastic modulus of 22.2 MPa. An rhBMP-7 (Olympus Biotech Corporation) formulation consisted of 3.5 mg of rhBMP-7 formulated with 1 g of purified bovine type 1 collagen carrier. The product was reconstituted with 3 ml of saline to form a paste, which was then transferred to the inner duct of the scaffold and the contact interfaces between bone and scaffold.

### EXAMPLE 2

### Study of Defect Regeneration in Load-Bearing Long Bones with mPCL-TCP Matrix and BMP-7

Sixty-four sheep of progressed age were chosen as study subjects because of their reduced intrinsic regenerative potential and their similarities to human bone regarding remodeling, turnover, and secondary osteon formation. Mid-diaphyseal tibial defects of 3-cm length were created and stabilized. The defects were left untreated/empty or were reconstructed with autologous bone graft (ABG), mPCL-TCP scaffolds, mPCL-TCP scaffolds and rhBMP-7, or mesenchymal stem cells (MSCs) seeded into mPCL-TCP scaffolds using autologous platelet-rich plasma (PRP) for cell delivery. The animals were not immobilized after surgery to create the defect, which was important to mimic the effects of a weight-bearing bone.

All animals were in good health and survived the 12-month in vivo study. No postoperative infections, implant failures, or macroscopic signs of foreign body reaction to the scaffolds occurred. One of eight animals treated with mPCL-TCP was excluded from analyses owing to a bone fracture through one of the proximal screw holes. One of eight animals treated with scaffold/rhBMP-7 showed evidence of a small and localized area of granulocytic infiltrate around the remnants of the collagen carrier.

To determine whether the selected method of defect fixation was suitable to protect scaffolds from excessive loads, before the transplantation study, we investigated the mechanical behavior of the fixation implant-bone scaffold construct on sheep cadaver tibiae. Biomechanical testing in vitro showed that, under an axial compression load of 500 N, the interfragmentary movement (IFM) in the defect containing a scaffold was 0.27 mm, giving an interfragmentary strain (IFS=IFM/gap size) of less than 1%. There was minimal difference in the IFM (0.21 mm) under compression when an empty defect (scaffold removed from defect) was tested. When subjected to torsion (7 N·m), the fixation implant-bone scaffold construct underwent a relative rotation of the bone fragments of 7.4°. Medial-lateral bending induced by an axial load of 100 N at an offset of 10 cm resulted in a shortening of the defect axially by 4.0 mm (IFS, 13%), with a bending angle of 1.9°.

X-ray analysis after 3 months confirmed the critical-sized nature of the defect, as shown by a union rate of 0% for the empty control defects. For the ABG and rhBMP-7 groups, all eight animals (100%) showed bone bridging the defect, but only three of eight (37.5%) showed bridging in the scaffold-only and MSC groups. In all groups, distinct bone formation along the fixation plate was observed, which is a phenomenon also observed in people.

Computed tomography (CT) values of total bone volume (BV) in the defect area were significantly higher with rhBMP-7 (8.6 cm³) when compared to all other scaffold-based groups. BV distribution along the defect's z axis showed a tendency toward more bone formation at the defect/bone interfaces. For the empty control defects, no biomechanical testing could be performed owing to a lack of bony bridging, leaving the defects filled with soft tissue only. Torsional stiffness values were significantly higher for the scaffold/rhBMP-7 group (at two concentrations of BMP: 1.75 and 3.5 mg) when compared to the mPCL-TCP scaffold-only group at 3 months. No significant difference in torsional moment or torsional stiffness was found between the ABG and the scaffold/rhBMP-7 groups, indicating that BMP-7 can induce bone of similar mechanical properties to ABG-mediated bone. However, a significant difference in torsional stiffness was determined for ABG and the scaffold/rhBMP-7 groups when compared to scaffold/MSC, suggesting that MSCs alone are not able to regenerate bone as well as BMP-7.

The calculated BVs, BV distribution, and mechanical properties correlate well with macroscopic findings in micro-CT (μCT) reconstructions and histological sections, and the corresponding animated three-dimensional (3D) μCT reconstructions of a representative sample of the control defects and the ABG, scaffold-only, and scaffold/rhBMP-7 groups.

Bone formation along the fixation plate—a common phenomenon in the clinic—and signs of cortex resorption in the proximity of the defect as indicated by a decreasing cortical density were observed in all animals (n=23). 3D μCT reconstructions showed only partial defect bridging with the scaffold only (n=7). In the ABG and scaffold/rhBMP-7 groups (n=8 each), signs of bone remodeling were evident after 12 months, such as restored long bone morphology characterized by dense cortical bone and a marrow cavity composed of cancellous bone. The amount of newly formed bone within each group varied considerably, as demonstrated in histological sections stained with Safranin Orange/von Kossa. No signs of scaffold degradation were evident.

Compared to the other treatments, overall mechanical strength (torsional moment) and torsional stiffness after 12 months were significantly higher when defects were augmented with the scaffold containing rhBMP-7. Improvements in strength and stiffness increased significantly over time for the ABG and scaffold/rhBMP-7 groups. For the scaffold-only group, torsional moment values increased minimally, but significantly, from 3 to 12 months, whereas torsional stiffness showed no significant change.

At 12 months, BV and polar moment of inertia (J_{z}) remained significantly lower in the scaffold-only group compared to the scaffold/rhBMP-7 group. When the scaffold-only group was compared to the ABG group, no difference was seen for BV. At 12 months, the scaffold/rhBMP-7-treated group exhibited higher BV and J_{z} values than the ABG group, suggesting that after 12 months, bone healing observed with scaffold/rhBMP-7 was superior compared to the gold standard autograft. For all three treatment groups, BV and J_{z} increased significantly over time.

Last, the mineralization of ABG- and scaffold/rhBMP-7-treated defects increased between months 3 and 12, whereas no significant changes were observed for the scaffold-only group.

BV distribution was determined using μCT in both the axial and the radial bone. Axial BV distribution was assessed in empty defects as well as in defects treated with ABG, scaffold only, or scaffold/rhBMP-7 by dividing the total length of the defect into three parts of equal length. In all treatment groups, there was a nonsignificant tendency toward greater bone formation in the proximal defect one-third, which is better vascularized, and more bone within the regions adjacent to the interfaces compared to the middle one-third, suggesting that defect regeneration is initiated by bone ingrowth at the defect regions proximate to the intact bone and subsequently advances toward the middle one-third.

We assessed radial bone distribution in scaffold-only- and scaffold/rhBMP-7-treated animals, looking at the inner duct, the wall, and the periphery. At 3 and 12 months, the amount of new bone formed in the periphery in both groups was comparable to within the scaffold wall and inner duct. Radial bone distribution per unit volume of scaffold wall and inner duct was homogeneous in the scaffold-only group after both 3 and 12 months. With the addition of rhBMP-7 to the scaffold, there was a trend toward greater bone formation in the inner duct after 3months, showing that BMP-7 locally increases bone formation mainly restricted to its site of application. At 12 months, however, significantly more bone was evident within the scaffold wall, indicating that BMP-7 drives bone remodeling and the restoration of the tubular long bone morphology.

The morphology of the newly formed bone with scaffold/rhBMP-7 was investigated on histology sections stained with Movat's pentachrome. An interface of old cortical bone and fibrolamellar bone with disorganized collagen fibers is characteristic for mammals when fast bone growth is required. At higher magnification, the vascularized, maturing bone tissue was observed to contain mineralized bone matrix, unmineralized osteoid, and mature osteocytes embedded in lacunae. The osteoid was located on the interface of mineralized bone and fibrous tissue and lined by bone-synthesizing osteoblasts and bone-resorbing osteoclasts. Blood vessels were embedded in soft tissue.

Backscattered electron (BSE) imaging was used to characterize bone morphology of contralateral tibiae. BSE imaging illustrates the largely plexiform bone morphology characteristic of ovine bone comprising a combination of woven and lamellar bones within which vascular plexuses are sandwiched. In the vicinity of the marrow cavity, secondary osteon formation was observed. Notably, secondary, osteonal remodeling in sheep normally does not take place until an average age of 7 to 9 years.

We claim:

1. An osteogenic implant, comprising:
a biodegradable scaffold comprising a matrix comprising polycaprolactone; and
a formulation carried by said scaffold, said formulation comprising BMP-7.

2. The osteogenic implant of claim 1, wherein said polycaprolactone is characterized by a molecular weight of about 80 kD.

3. The osteogenic implant of claim 1, wherein said scaffold further comprises ceramic particles.

4. The osteogenic implant of claim 3, wherein said ceramic particles comprise a calcium phosphate.

5. The osteogenic implant of claim 4, wherein said calcium phosphate comprises tricalcium phosphate.

6. The osteogenic implant of claim 3, wherein the amount of ceramic particles within said scaffold is within the range of about 20 weight percent to about 80 weight percent.

7. The osteogenic implant of claim 1, wherein said matrix comprises polymeric fibers.

8. The osteogenic implant of claim 7, wherein said fibers are arranged in a 0/90° pattern within said scaffold.

9. The osteogenic implant of claim 7, wherein said fibers are arranged in a 0/60/120° pattern within said scaffold.

10. The osteogenic implant of claim 7, wherein said fibers are arranged in a 0/72/144/36/108° pattern within said scaffold.

11. The osteogenic implant of claim 7, wherein said fibers have a diameter in the range of about 260 microns to about 370 microns.

12. The osteogenic implant of claim 7, wherein said fibers are arranged within said scaffold such that the average pore size between said fibers is in the range of about 200 microns to about 700 microns.

13. The osteogenic implant of claim 12, wherein the porosity of said scaffold is in the range of about 30% to about 80%.

14. The osteogenic implant of claim 13, wherein said porosity is interconnected porosity.

15. The osteogenic implant of claim 7, wherein said scaffold is a three dimensional structure having a lumen extending at least partially through said scaffold, said lumen having an opening on a surface of said scaffold.

16. The osteogenic implant of claim 15, wherein said formulation is at least partially located within said lumen.

17. The osteogenic implant of claim 7, wherein said scaffold is a three dimensional structure in the shape of an interbody spine fusion cage.

18. The osteogenic implant of claim 1, wherein said formulation further comprises bovine collagen.

19. The osteogenic implant of claim 1, wherein said formulation is in a form selected from the group consisting of a gel, a paste, a putty, a film, and granules.

20. An osteogenic implant, comprising:
a three-dimensional biodegradable scaffold having a lumen extending at least partially therethrough, said scaffold having a porosity with a range of 60% to 80% and comprising a matrix comprising polymeric fibers comprising polycaprolactone, said fibers having an average diameter with a range of 200 microns to 400 microns, said fibers arranged in a 0/90° pattern within said scaffold, said scaffold further comprising 20 weight percent to 30 weight percent of particles comprising a calcium phosphate; and
a formulation carried by said scaffold, said formulation comprising BMP-7 and bovine collagen.

21. The osteogenic implant of claim 20, wherein the porosity of said scaffold is about 70%.

22. The osteogenic implant of claim 20, wherein the average diameter of said fibers is about 300 microns.
